(19) United States
(12) Patent Application Publication
(10) Pub. No.: US 2015/0239819 A1
(43) Pub. Date: Aug. 27, 2015

(54) METHOD FOR MANUFACTURING METAL PRECURSOR AND METHOD FOR MANUFACTURING METAL INK BY USING METAL PRECURSOR

(71) Applicant: SAMSUNG FINE CHEMICALS CO., LTD, Ulsan (KR)

(72) Inventors: Sung Soon Kim, Gyeonggi-do (KR); Eui Hyun Ryu, Daejeon (KR); Chan Hyuk Park, Seoul (KR); Dong Woo Kim, Daejeon (KR); Kyung Yol Yon, Gyeonggi-do (KR)

(73) Assignee: SAMSUNG FINE CHEMICALS CO., LTD, Ulsan (KR)

(21) Appl. No.: 14/422,406

(22) PCT Filed: May 9, 2013
(86) PCT No.: PCT/KR2013/004105

§ 371 Date: Feb. 19, 2015

(30) Foreign Application Priority Data
Aug. 23, 2012 (KR) 10-2012-0092317

(51) Int. Cl. C07C 51/41; C09D 11/52

(52) U.S. Cl. CPC C07C 51/418; C09D 11/52

(57) ABSTRACT

A method of preparing a metal precursor and a method of preparing a metal ink using the same are provided. The method of preparing a metal precursor and the method of preparing a metal ink using the same can be useful in facilitating separation and purification of a precursor from impurities formed during precursor synthesis by allowing a metal precursor to form a suboligomeric aggregate as the metal precursor is synthesized using a fatty acid having a substituent at an α position, and also enhancing solubility, enabling low-temperature sintering and improving coating film physical properties and electrical properties of the final metal ink.

### TECHNICAL FIELD

The present invention relates to a method of preparing a metal precursor, and a method of preparing a metal ink using the metal precursor prepared by the same. More particularly, the present invention relates to a method of preparing a metal precursor capable of facilitating separation and purification of the precursor from impurities formed during precursor synthesis by forming a suboligomeric aggregate upon synthesis of the precursor using a fatty acid having a substituent at an α position and precipitating the suboligomeric aggregate by drastically reducing solubility in a polar solution phase, and also to a method of preparing a metal precursor ink capable of exhibiting high solubility in various solvents, enabling low-temperature sintering and improving coating film physical properties and electrical properties of the final metal ink by inhibiting formation of a polymeric coordination compound during preparation of the metal ink due to a steric hindrance effect by a functional group present at the α position.

### BACKGROUND ART

A metal ink has been used for various products such as a conductive ink, an electromagnetic wave shielding agent, a reflective film forming material, an antibacterial agent, etc. In particular, conductive inks are used due to current regulations on use of lead in electric/electronic circuits, used for low-resistivity metal interconnections, printed circuit boards (PCBs), flexible printed circuit boards (FPCBs), antennas for radio frequency identification (RFID) tags and electromagnetic wave shielding materials, and are useful when a metal pattern is required or electrodes are simply formed in the field of new applications such as plasma display panels (PDPs), liquid crystal displays (TFT-LCDs), organic light emitting diodes (OLEDs), flexible displays and organic thin film transistors (OTFTs), and thus attention has been increasingly paid to the conductive inks. With the tendency toward highly functional and very thin electronic products, metal particles used in the electronic products are gradually becoming finer in size.

In general, a metal ink may be prepared by dissolving a metal precursor in an organic solvent. Here, the metal precursor is prepared by means of synthesis. In this case, since reaction by-products and unreacted materials are included in a reaction solution in the course of synthesis, physical properties of the final ink may be affected.

As an alternative to solve this problem, a method of synthesizing a metal precursor using a linear fatty acid has been presented. The method has an advantage in that a metal precursor may be readily separated and purified since a precipitate of the metal precursor is very easily formed due to a change in polarity before/after reaction and formation of a polymeric aggregate. However, the method has a problem in that the metal precursor has very low solubility due to formation of a polymeric coordination compound. Also, ammonia-based bases applied to enhance the solubility may degrade physical properties after sintering.

Accordingly, the present inventors have conducted much research to find a way to facilitate separation and purification of the synthesized metal precursor and also enhance solubility of the synthesized metal precursor, and thus found that, when a metal precursor is synthesized using a fatty acid having a substituent at an α position, the metal precursor can be easily separated and purified, and also can function to enhance solubility in various solvents, enable low-temperature sintering and improve coating film physical properties and electrical properties of the final metal ink upon formation of a suboligomeric aggregate due to a steric hindrance effect caused by the substituent present at the α position. Therefore, the present invention has been completed based on these facts.

### DISCLOSURE

### Technical Problem

The present invention is directed to a method of preparing a metal precursor capable of being used to prepare a metal ink which can be easily separated and purified after synthesis, have high solubility, be sintered at a low temperature, and exhibit excellent coating film physical properties and electrical properties.

Also, the present invention is directed to a method of preparing a metal ink from a metal precursor which can be easily separated and purified after synthesis, have high solubility, be sintered at a low temperature, and exhibit excellent coating film physical properties and electrical properties.

### Technical Solution

According to an aspect of the present invention, there is provided a method of preparing a metal precursor for preparing a metal ink. Here, the method includes forming a metal precursor having a substituent at an α position by reaction of a metal salt with a fatty acid having a substituent at an α position in an organic solvent, and separating the metal precursor having a substituent at an α position.

In the method of preparing a metal precursor according to the present invention, the fatty acid having a substituent at an α position may have a structure represented by the following Formula 1.

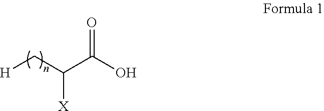

In Formula 1, X represents an alkyl group having 1 to 6 carbon atoms, or a halogen, and n is an integer ranging from 0 to 23.

In the method of preparing a metal precursor according to the present invention, the formation of the metal precursor may particularly include (i) preparing a fatty acid solution by dissolving a fatty acid having a substituent at an α position in an organic solvent, and (ii) mixing a metal salt solution with the fatty acid solution to allow the metal salt solution to react with the fatty acid solution.

Also, the solvent may be at least one selected from the group consisting of organic solvents such as CH₂CN, CH₃OH, CH₃CH₂OH, THF, DMSO, DMF, 1-methoxy-2-propanol, 2,2-dimethoxypropanol, 4-methyl-2-pentanone and dibutyl ether, and water.

Also, in the reaction of the metal salt solution with the fatty acid solution by dropping the metal salt solution in the fatty acid solution, first, the metal salt solution is prepared by dissolving the metal salt in the organic solvent. Here, the solvent that may be used herein may include at least one selected from the group consisting of organic solvents such as CH₂CN, CH₃OH, CH₃CH₂OH, THF, DMSO, DMF, 1-methoxy-2-propanol, 2,2-dimethoxypropanol, 4-methyl-2-pentanone and dibutyl ether, and water. Then, the metal salt solution may be allowed to react with the fatty acid solution by dropping the metal salt solution in the fatty acid solution. In this case, intense agitation may simultaneously accompany the dropping.

In this case, the fatty acid solution may further include at least one base selected from the group consisting of KOH, NaOH, NH₃, NH₂CH₃, NH₄OH, NH(CH₃)₂, N(CH₃)₃, NH₂Et, NH(Et)₂, NEt₃ and Ca(OH)₂.

Metal ions used in the metal salt solution may be selected from the group consisting of Ag, Pd, Rh, Cu, Pt, Ni, Fe, Ru, Os, Mn, Cr, Mo, Au, W, Co, Ir, Zn and Cd.

In addition, the metal salt reacting with the fatty acid may be in the form of a nitride.

According to another aspect of the present invention, there is provided a method of preparing a metal ink. Here, the metal ink is prepared by dissolving a metal precursor having a substituent at an α position in an organic solvent, and the metal precursor is formed by allowing a fatty acid having a substituent at an α position to react with a metal in an organic solvent.

The organic solvent used to dissolve the metal precursor may be at least one selected from the group consisting of THF, xylene, toluene, methylene chloride, CH₃OH, CH₃CH₂OH, CH₃CH₂CH₂OH and DMSO.

Furthermore, to further homogenize the metal ink, the method may further include performing supersonic agitation, eddy current agitation, mechanical agitation or ball mill treatment on the metal ink.

### Advantageous Effects

The method of preparing a metal precursor according to the present invention and the method of preparing a metal ink using the same have the following effects.

First, the metal precursor can be easily separated and purified when the metal precursor forms a suboligomeric aggregate using a fatty acid having a substituent at an α position.

Second, the fatty acid having a substituent at an α position may be used to form a suboligomeric aggregate due to a steric hindrance effect caused by the substituent, thereby enhancing solubility in various solvents

Third, in the method of preparing a metal precursor according to the present invention, the metal precursor that can be sintered at a low temperature can be prepared by adjusting a length of the metal precursor.

Fourth, since the metal precursor according to the present invention has phase transition behavior such as a liquid crystalline phase at a temperature lower than a conventional sintering temperature, a structure in which metals are well aligned can be formed to improve physical properties of a coating film, and conductivity may be improved with an increase in contact probability between the metals.

### DESCRIPTION OF DRAWING

The above and other objects, features and advantages of the present invention will become more apparent to those of ordinary skill in the art by describing in detail exemplary embodiments thereof with reference to the accompanying drawings, in which:

FIG. 1 is a scheme showing a process of synthesizing a metal precursor having a substituent at an α position according to one exemplary embodiment of the present invention;

FIG. 2 is a schematic diagram showing a change in solubility according to the substituent present in the α position of the metal precursor according to one exemplary embodiment of the present invention;

FIG. 3 is a schematic diagram showing phase transition behavior such as a liquid crystalline phase of a metal precursor at a low sintering temperature according to one exemplary embodiment of the present invention;

FIG. 4 shows the analysis data confirming oligomeric formation of a Ag precursor prepared according to one exemplary embodiment of the present invention;

FIG. 5 shows the thermogravimetric analysis (TGA) results capable of confirming thermal behavior of the Ag precursor prepared according to one exemplary embodiment of the present invention; and

FIG. 6 shows the DSC results capable of confirming the thermal behavior of the Ag precursor prepared according to one exemplary embodiment of the present invention.

### BEST MODE

Exemplary embodiments of the present invention will be described in detail below with reference to the accompanying drawings. While the present invention is shown and described in connection with exemplary embodiments thereof, it will be apparent to those skilled in the art that various modifications can be made without departing from the scope of the invention.

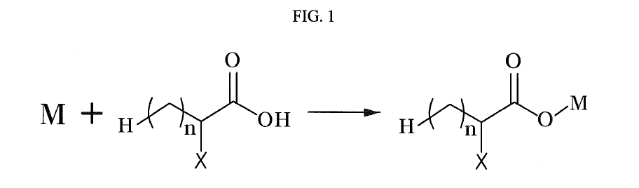

FIG. 1 is a scheme schematically showing a procedure of synthesizing a metal precursor having a substituent at an α position according to one exemplary embodiment of the present invention.

Referring to FIG. 1, the synthesis of the metal precursor according to the present invention is performed by allowing a metal to react with a fatty acid having a substituent at an α position in the presence of an organic solvent and a base catalyst to synthesize a metal precursor having a substituent at an α position.

More particularly, in the present invention, the formation of the metal precursor includes preparing a fatty acid solution by dissolving a fatty acid having a substituent at an α position in an organic solvent; mixing a metal salt solution with the fatty acid solution to allow the metal salt solution to react with the fatty acid solution; and forming a metal precursor precipitate from the mixed solution.

In the preparation of the fatty acid solution by dissolving the fatty acid having a substituent at an α position in the organic solvent, since the fatty acid having a substituent at an α position has the substituent present at an α position, the fatty acid may control formation of a suboligomeric aggregate caused by a steric hindrance effect upon synthesis of the metal precursor. As a result, very low solubility of the metal precursor, which may be caused when the metal precursor is synthesized using a linear fatty acid, may be solved by formation of a polymeric aggregate.

The fatty acid having a substituent at an α position may have a structure represented by the following Formula 1.

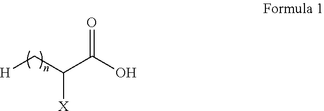

In Formula 1, X represents an alkyl group having 1 to 6 carbon atoms, or a halogen, and n is an integer ranging from 0 to 23.

The preferred fatty acid may be 2-methyl heptanoic acid, 2-methyl hexanoic acid, 2,2-dimethylbutyric acid, 2-ethylhexanoic acid, hexanoic acid, acrylic acid, or isobutyric acid.

Also, the solvent may be at least one selected from the group consisting of organic solvents such as CH₂CN, CH₃OH, CH₃CH₂OH, THY, DMSO, DMF, 1-methoxy-2-propanol, 2,2-dimethoxypropanol, 4-methyl-2-pentanone and dibutyl ether, and water.

The fatty acid solution may further include at least one base selected from the group consisting of KOH, NaOH, NH₃, NH₂CH₃, NH₄OH, NH(CH₃)₂, N(CH₃)₃, NH₂Et, NH(Et)₂, NEt₃ and Ca(OH)₂.

In the reaction of the metal salt solution with the fatty acid solution by dropping the metal salt solution in the fatty acid solution, first, the metal salt solution is prepared by dissolving a metal salt in an organic solvent. Here, the solvent used to dissolve the metal salt may be at least one selected from the group consisting of organic solvents such as CH₂CN, CH₃OH, CH₃CH₂OH, THF, DMSO, DMF, 1-methoxy-2-propanol, 2,2-dimethoxypropanol, 4-methyl-2-pentanone and dibutyl ether, and water.

Next, the metal salt solution is dropped in the fatty acid solution so as to react with the fatty acid solution. In this case, intense agitation may simultaneously accompany the dropping. Metal ions of the metal salt may be selected from the group consisting of Ag, Pd, Rh, Cu, Pt, Ni, Fe, Ru, Os, Mn, Cr, Mo, Au, W, Co, Ir, Zn and Cd, and Ag is more preferred. All of a nitride, an oxide, a sulfide and a halide may be used as an anionic material of the metal salt. Among these, the anionic material of the metal salt is preferably in the form of a nitride.

The fatty acid solution and the metal solution may be mixed at a weight ratio of 1:1 to 10:1 or 1:10. The reaction may be performed at room temperature.

In the formation of the metal precursor precipitate from the mixed solution, the mixed solution in which dropping of the metal salt solution is completed may be further stirred for 1 to 30 minutes to form a precipitate.

In the separation of the precipitate, the precipitate may be removed using conventional separation methods known in the related art. More particularly, a method such as filtration or recrystallization may be used herein.

Subsequently, the separated precipitate may be washed several times with at least one selected from the group consisting of organic solvents used to synthesize the precipitate, for example, CH₂CN, CH₃OH, CH₃CH₂OH, THF, DMSO, DMF, 1-methoxy-2-propanol, 2,2-dimethoxy propane, 4-methyl-2-pentanone and dibutyl ether, and water, and then dried to form a metal precursor having a final structure represented by the following Formula 2.

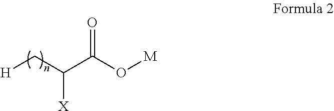

In Formula 2, X represents an alkyl group having 1 to 6 carbon atoms, or a halogen, M is selected from the group consisting of Ag, Pd, Rh, Cu, Pt, Ni, Fe, Ru, Os, Mn, Cr, Mo, Au, W, Co, Ir, Zn and Cd, and n is an integer ranging from 0 to 23.

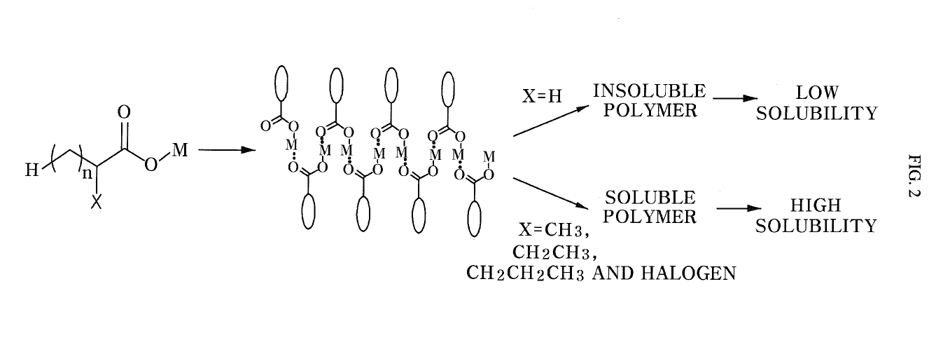

As shown in FIG. 2, the metal precursor prepared using the fatty acid forms a coordination compound, and exhibits a significant difference in solubility according to the kind of substituent. That is, in the case of the metal precursor using the linear fatty acid, an insoluble polymer is formed to exhibit very low solubility. On the other hand, the metal precursor prepared using a fatty acid having a substituent, such as a methyl, ethyl or propyl group, or a halogen, at an α position according to the present invention may have high solubility since the metal precursor forms a soluble suboligomeric aggregate.

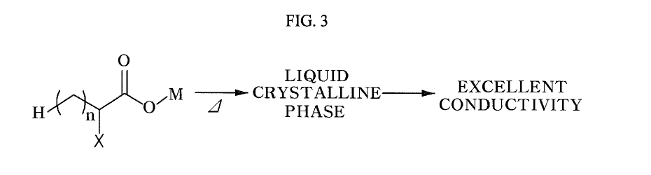

Also, the metal precursor prepared according to the present invention has a liquid crystalline phase at a temperature lower than a conventional sintering temperature, for example, a temperature of less than 250° C., as shown in FIG. 3. In this case, a structure in which metals are well aligned may be formed to improve physical properties of a coating film, and conductivity may be improved with an increase in contact probability between the metals.

The present invention provides a method of preparing a metal ink prepared by dissolving the metal precursor prepared using the above-described method in an organic solvent.

The organic solvent used to prepare the metal precursor ink may be at least one selected from the group consisting of an ether selected from THF, ethyl ether, propyl ether and MEK; a benzene selected from xylene, toluene, ethylbenzene and benzene; an alcohol selected from methanol, ethanol, butanol, propanol, ethylene glycol and propylene glycol; a chloride selected from methylene chloride and chloroform; dimethylsulfoxide (DMSO); a nitride selected from dimethylformamide (DMF) and diethylformamide (DEF); and an alkyl selected from hexane, pentane and butane.

Also, the method according to the present invention may further include mixing an additive for adjusting the physical properties. In this case, physical properties of the final ink obtained by adding the additive required for a coating or printing process may be adjusted. General kinds of additives known in the related art may be widely used in a general content range. In the case of the additive used herein, for example, an amine, especially, NH₃, NH(CH₃)₂, N(CH₃)₃, NH₂Et, NH(Et)₂ or NEt₃, may be added at a content of approximately 0.1 to 50% by weight, and a surfactant such as polyvinylpyrrolidone (PVP), polyacrylic acid (PAA), sodium dodecyl sulfate (SDS), Tween 20™ or DowFax™ may be added as a dispersion stabilizer at a content of approximately 0.1% to 5% by weight, based on the total weight of the final ink. Also, a thickener may also be added at a content of approximately 0.1% to 5% by weight, based on the total weight of the final ink.

To further homogenize the metal ink, the method may also further include performing supersonic agitation, eddy current agitation, mechanical agitation or ball mill treatment on the metal ink. For example, the supersonic agitation may be performed for approximately 10 minutes to 2 hours at 5 to 50 Hz, the eddy current agitation may be performed for approximately 2 to 4 hours at 100 to 1,500 rpm, and the ball mill treatment may be performed by introducing balls and a solution at a weight ratio of 1:1 and stirring the solution for approximately 4 to 12 hours.

Hereinafter, the present invention will be described in further detail with reference to the following Examples. However, it should be understood that the description presented herein is not intended to limit the scope of the present invention.

### Example 1

### Synthesis of Ag Precursor

1.7 g of 2-methyl hexanoic acid was put into a 250 ml flask, and dissolved in 84 ml of a polar organic solvent, CH₃CN, and 2.7 g of NEt₃ was added as a base. Thereafter, 1.4 g of AgNO₃ was put into another 250 ml flask, and dissolved in 84 ml of CH₃CN. The AgNO₃ solution was slowly dropped in the 2-methyl hexanoic acid solution at a rate of 700 ml/hr while vigorously stirring. The mixed solution in which addition of the AgNO₃ solution was completed was stirred for 20 minutes, and a precipitate was separated, washed twice with an organic solvent (CH₃CN), and then dried to form approximately 2.0 g of a Ag precursor (Ag-2-methyl hexanoate).

### Example 2

### Synthesis of Ag Ink

0.6 g of Ag-2-methyl hexanoate was dissolved in 3.6 g of xylene. Thereafter, an amine (NH₃) that was a catalyst, and polyvinylpyrrolidone that was a dispersion stabilizer were added as additives at a content of 5%, based on the total weight, and uniformly mixed by supersonic agitation for an hour at 30 Hz to prepare a Ag ink.

### Experimental Example 1

Oligomeric formation and thermal behavior of the Ag precursor prepared in Example 1 were confirmed using the following methods.

Confirmation of Oligomeric Formation

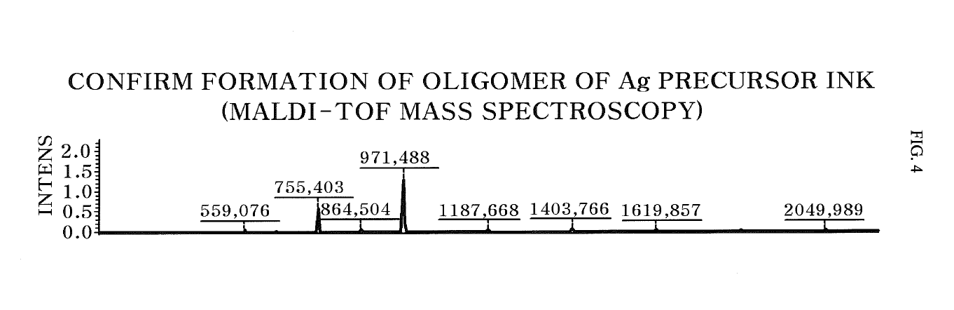

Oligomeric formation of the Ag precursor prepared in Example 1 was confirmed using a MALDI-TOF mass spectrometer. The results are shown in FIG. 4.

Confirmation of Thermal Behavior

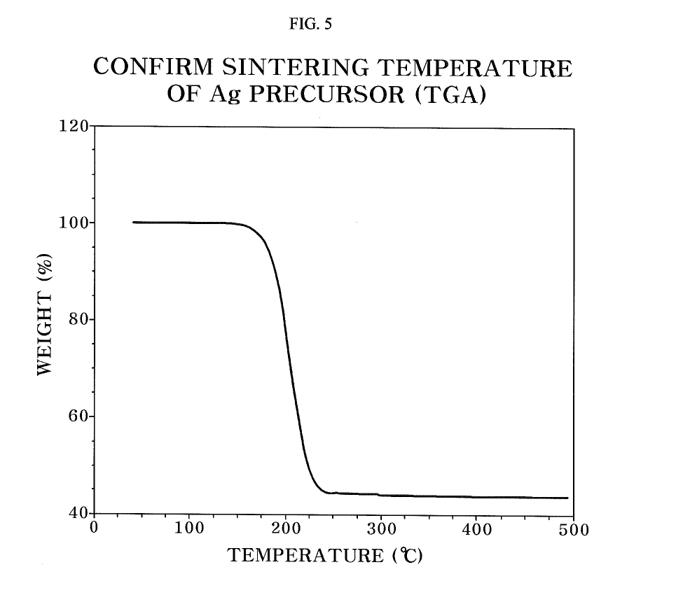

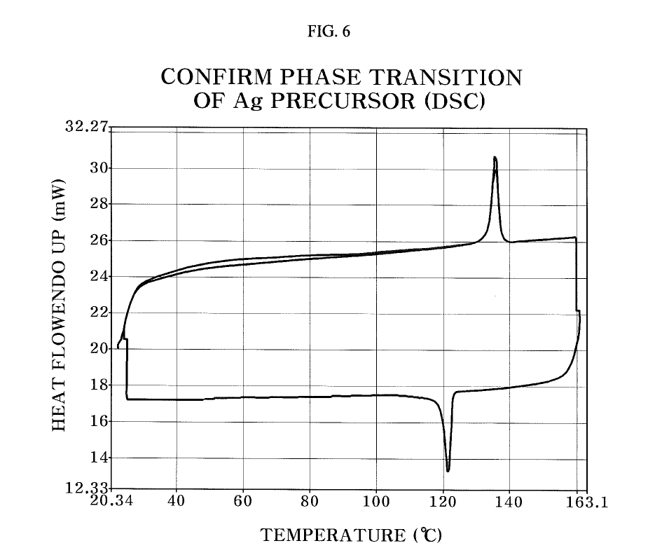

Thermal behavior of the Ag precursor prepared in Example 1 was confirmed using a sintering temperature and a phase transition obtained through TGA and DSC. The results are shown in FIGS. 5 and 6.

FIG. 5 is a graph showing how the X axis in the TGA data is plotted against a mass of the Y axis according to a change in temperature. When it was assumed that the Ag precursor was present at a content of 100% by weight, a sudden decrease in mass of the Ag precursor was observed around 200° C., and the Ag precursor converged again at a temperature of nearly 200° C. This indicates that the Ag precursor starts to decompose around 200° C. and its sintering is completed around 240° C.

FIG. 6 shows the DSC data confirming whether the Ag precursor is metallic by measuring a phase transition temperature of the Ag precursor. Referring to FIG. 6, it could be seen that peaks were observed around 135° C. upon both of first heating and second heating, and a third phase was observed before sintering at this temperature.

### Experimental Example 2

The Ag ink prepared in Example 2 was coated or printed, and sintered at 250° C. for 20 minutes. Thereafter, the coated coating film was measured for surface resistivity using a 4-point probe. As a result, it was revealed that the coating film had a specific resistivity of 7μΩ·cm.

1. A method of preparing a metal precursor, comprising:
forming a metal precursor having a substituent at an α position by reaction of a metal salt with a fatty acid having a substituent at an α position in an organic solvent; and
separating the metal precursor having a substituent at an α position.

2. The method of claim 1, wherein the fatty acid having a substituent at an α position has a structure represented by the following Formula 1: 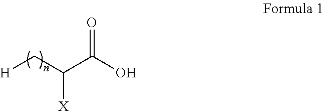
wherein X represents an alkyl group having 1 to 6 carbon atoms or a halogen, and n is an integer ranging from 0 to 23.

3. The method of claim 1, wherein the formation of the metal precursor comprises:
(i) preparing a fatty acid solution by dissolving a fatty acid having a substituent at an α position in an organic solvent; and
(ii) mixing a metal salt solution with the fatty acid solution to allow the metal salt solution to react with the fatty acid solution.

4. The method of claim 1, wherein the solvent is at least one selected from the group consisting of CH₂CN, CH₃OH, CH₃CH₂OH, THF, DMSO, DMF, 1-methoxy-2-propanol, 2,2-dimethoxypropanol, 4-methyl-2-pentanone and dibutyl ether, and water.

5. The method of claim 1, wherein, in the formation of the metal precursor, the fatty acid solution further comprises at least one base selected from the group consisting of KOH, NaOH, NH₃, NH₂CH₃, NH₄OH, NH(CH₃)₂, N(CH₃)₃, NH₂Et, NH(Et)₂, NEt₃ and Ca(OH)₂.

6. The method of claim 1, wherein the metal precursor has a structure represented by the following Formula 2: 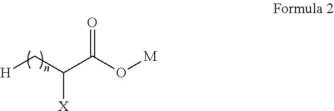
wherein X represents an alkyl group having 1 to 6 carbon atoms, or a halogen, M is selected from the group consisting of Ag, Pd, Rh, Cu, Pt, Ni, Fe, Ru, Os, Mn, Cr, Mo, Au, W, Co, Ir, Zn and Cd, and n is an integer ranging from 0 to 23.

7. A method of preparing a metal ink which is prepared by dissolving the metal precursor prepared by the method defined in claim 1 in an organic solvent.

8. The method of claim 7, wherein the organic solvent is at least one selected from the group consisting of an ether selected from THF, ethylether, propylether and MEK; a benzene selected from xylene, toluene, ethylbenzene and benzene; an alcohol selected from methanol, ethanol, butanol, propanol, ethylene glycol and propylene glycol; a chloride selected from methylene chloride and chloroform; dimethylsulfoxide (DMSO); a nitride selected from dimethylformamide (DMF) and diethylformamide (DEF); and an alkyl selected from hexane, pentane and butane.

9. The method of claim 7, further comprising:
homogenizing the metal ink by applying supersonic agitation, eddy current agitation, mechanical agitation or ball mill treatment to the metal ink.
